(19) United States
(12) Patent Application Publication
(10) Pub. No.: US 2013/0217046 A1
(43) Pub. Date: Aug. 22, 2013

(54) METHOD FOR DETERMINING PREDISPOSITION TO ESOPHAGEAL-RELATED DISORDERS

(71) Applicants: Wancai Yang, Chicago, IL (US); Li-Dong Wang, Xinxiang (CN)

(72) Inventors: Wancai Yang, Chicago, IL (US); Li-Dong Wang, Xinxiang (CN)

(73) Assignee: The Board of Trustees of the Univer. of Illinois, Urbana, IL (US)

(21) Appl. No.: 13/805,681

(22) PCT Filed: Jun. 23, 2011
(86) PCT No.: PCT/US11/41659

§ 371 Date: Dec. 20, 2012

Related U.S. Application Data
(60) Provisional application No. 61/357,780, filed on Jun. 23, 2010.

(51) Int. Cl. C12Q 1/68

(52) U.S. Cl. CPC C12Q 1/6827
USPC 435/7.92; 435/6.11; 435/6.14; 436/501; 530/327

(57) ABSTRACT

Provided herein are methods and materials for diagnosing an esophageal or esophageal-related disorder, or a predisposition for such disorders, in a subject. The methods center on detecting a genetic or protein esophageal marker. An esophageal marker has been identified in the PLCE1 gene and may be useful in predicting disease progression and assessing the subject's response to therapy.

### CROSS-RELATED APPLICATIONS

The present application claims the benefit of the filing date of provisional application 61/357,780, filed on Jun. 23, 2010, which is incorporated by reference in its entirety.

### FEDERALLY SPONSORED RESEARCH

This invention was made with government support under grant number R01CA112081 awarded by the National Institutes of Health and the National Cancer Institute. The government has certain rights in the invention.

### FIELD OF THE INVENTION

The present invention relates to a single nucleotide polymorphism in the phospholipase C epsilon 1 gene (PLCE1) and its association with disorders of the esophagus.

### BACKGROUND

Gastro esophageal reflux disease can be uncomfortable, with burning in the chest, intolerance to big meals, asthmatic type symptoms and interruption of sleep. In some cases, it can lead to more dangerous symptoms. The acid can permanently damage the lining of the esophagus, and thereby creating a potential for pre-cancerous changes in the esophagus, such as esophagitis and/or esophageal cancer.

An esophageal disorder or esophageal related disorder may be suspected for a variety of reasons, but the definitive diagnosis of most disorders is traditionally confirmed by upper endoscopies, gastrointestinal function tests such as esophageal manometry, pH studies, esophageal impedance-pH testing, and/or histological examination of cancerous cells. With respect to cancer, tissue diagnosis may indicate the type of cell that is proliferating, its histological grade, genetic abnormalities, and other features of the tumor. Together, this information is useful to evaluate the prognosis of the patient and to choose the best treatment.

Early assessment of the esophageal disorder or esophageal-related disorder may present the best opportunity for treatment intervention. With the development of genetic testing, it is possible to identify genetic markers that will be indicative of a propensity to develop disease or indicative of a disease state. There remains a need to identify one or more genetic markers that are associated with esophageal disorders or esophageal-related disorders, such as cancer and/or esophagitis. These genetic markers may represent allelic variants, which may be useful in diagnosing a disease type, and whose products may be targeted for early intervention therapy.

### SUMMARY OF THE INVENTION

Provided herein is a method for determining a subject's predisposition for an esophageal disorder or esophageal-related disorder. The method may comprise providing a nucleic acid-containing sample obtained from a subject and determining whether the sample comprises an esophageal marker (e-marker), wherein the present of the marker indicates that the subject has a predisposition for an esophageal disorder or an esophageal-related disorder. The e-marker may be SEQ ID NO:1 or a fragment thereof. The fragment may comprise a contiguous nucleotides from SEQ ID NO:1, wherein the contiguous sequence contains the guanine at position 401 of SEQ ID NO:1. The method may further comprise determining the presence of one or more other markers, such as TFF2, HE4, LGALS3, IL1RN, TRIP133, FIGNL1, CRIP1, S100A4, EXOSC8, EXPI, BRRN1, NELF, EREG, TMEM40 and/or TMEM109. The esophageal disorder may be esophageal cancer or esophagitis. The esophageal cancer may be squamous cell carcinoma or esophagus-cardia gastric adenocarcinoma. The subject who is predisposed to esophagitis may not have cancer. The esophageal related disorder may be a cancer such as head and neck cancer, throat cancer, gastric cancer and/or mouth cancer. The e-marker may be detected by amplifying a nucleic acid comprising the marker and detecting the amplified nucleic acid, thereby detecting the marker. The nucleic acid may be amplified using pairs of primers, such as SEQ ID NOs:8 and 9; SEQ ID NOs: 10 and 11; or SEQ ID NOs:12 and 13. The detection may be accomplished with direct sequencing or hybridizing an oligonucleotide probe to the amplified product. The probe may be labeled with a detectable label. The oligonucleotide may comprise SEQ ID NO:1, or a fragment thereof, such as a fragment that comprises between 10 and 100 contiguous nucleotides of SEQ ID NO:1, and wherein the contiguous sequence contains the guanine at position 401 of SEQ ID NO:1. The fragment may be SEQ ID NO:2 or SEQ ID NO:14.

Also provided herein is an antibody-based method for determining a subject's predisposition for an esophageal disorder or esophageal-related disorder. The method may comprise contacting an antibody that specifically binds to a polypeptide encoded by the SEQ ID NO:1, or a fragment thereof, with a sample, thereby forming a complex between the antibody and the polypeptide; and detecting the presence of the complex, thereby detecting the marker. The presence of the marker indicates that the subject has a predisposition for a disorder of the esophagus or an esophageal-related disorder. The fragment may comprise between 15 and 86 amino acids of SEQ ID NO:3, wherein the contiguous sequence contains the arginine at position 53 of SEQ ID NO:3.The fragment may be SEQ ID NO:3, SEQ ID NO:4 or SEQ ID NO:7. The antibody may be labeled with a detectable label.

Also provided herein is a method for determining whether a subject has an esophageal disorder or esophageal-related disorder. The method may comprise providing a nucleic acid-containing sample obtained from a subject and determining whether the sample comprises an esophageal marker (e-marker), wherein the present of the marker indicates that the subject has an esophageal disorder or an esophageal-related disorder. The e-marker may be SEQ ID NO:1 or a fragment thereof. The fragment may comprise a contiguous nucleotides from SEQ ID NO:1, wherein the contiguous sequence contains the guanine at position 401 of SEQ ID NO:1. The method may further comprise determining the presence of one or more other markers, such as TFF2, HE4, LGALS3, IL1RN, TRIP133, FIGNL1, CRIP1, S100A4, EXOSC8, EXPI, BRRN1, NELF, EREG, TMEM40 and/or TMEM 109. The esophageal disorder may be esophageal cancer or esophagitis. The esophageal cancer may be squamous cell carcinoma or esophagus-cardia gastric adenocarcinoma. The subject who is predisposed to esophagitis may not have cancer. The esophageal related disorder may be a cancer such as head and neck cancer, throat cancer, gastric cancer and/or mouth cancer. The e-marker may be detected by amplifying a nucleic acid comprising the marker and detecting the amplified nucleic acid, thereby detecting the marker. The nucleic acid may be amplified using pairs of primers, such as SEQ ID NOs:8 and 9; SEQ ID NOs: 10 and 11; or SEQ ID NOs:12 and 13. The detection may be accomplished with direct sequencing or hybridizing an oligonucleotide probe to the amplified product. The probe may be labeled with a detectable label. The oligonucleotide may comprise SEQ ID NO:1, or a fragment thereof, such as a fragment that comprises between 10 and 100 contiguous nucleotides of SEQ ID NO:1, and wherein the contiguous sequence contains the guanine at position 401 of SEQ ID NO:1. The fragment may be SEQ ID NO:2 or SEQ ID NO:14.

Also provided herein is an antibody-based method for determining whether a subject has an esophageal disorder or esophageal-related disorder. The method may comprise contacting an antibody that specifically binds to a polypeptide encoded by the SEQ ID NO:1, or a fragment thereof, with a sample, thereby forming a complex between the antibody and the polypeptide; and detecting the presence of the complex, thereby detecting the marker. The presence of the marker indicates that the subject has a disorder of the esophagus or an esophageal-related disorder. The fragment may comprise between 15 and 86 amino acids of SEQ ID NO:3, wherein the contiguous sequence contains the arginine at position 53 of SEQ ID NO:3.The fragment may be SEQ ID NO:3, SEQ ID NO:4 or SEQ ID NO:7. The antibody may be labeled with a detectable label.

Also provided herein is a kit that may comprise a nucleic acid sample collecting means; a means for determining the presence of a esophageal marker in a nucleic acid; and a control sample comprising polymorphic DNA, wherein the polymorphic DNA is rs2274223 or a fragment thereof.

Also provided herein is a kit for determining the presence of an esophageal marker in a protein comprising a sample collecting means; a means for determining the presence of an esophageal marker in a protein; and a control sample comprising a polypeptide encoded by SEQ ID NO:1, or a fragment thereof.

Also provided herein is an isolated peptide consisting of SEQ ID NO:4.

### BRIEF DESCRIPTION OF THE DRAWINGS

FIG. 1 shows the PLCE1 genotypes (A) and an example of G allelic imbalance in cancer tissue (C) compared to normal (B).

FIG. 2 shows the AG genotype is associated with esophagitis, cancer differentiation and lymph node metastasis.

FIG. 3 shows the increase of PLCE1 mRNA was linked to AG genotype in human esophageal cancer cell lines.

FIG. 4 shows PLCE 1 protein and mRNA levels were increased in esophageal cancer tissues (esophageal squamous cell carcinoma—SCC), assayed by immunohistochemical staining and qRT-PCR.

FIG. 5 shows PLCE1 (also known as “PLCe”) schematic. The PLCe gene is located in chromosome 10 and has 32 exons. SNP rs2274223 is located in exon 26. A5780G mutation (CAC→CGC) introduces a histidine (H) to arginine (R) mutation in calcium binding domain (C2 domain).

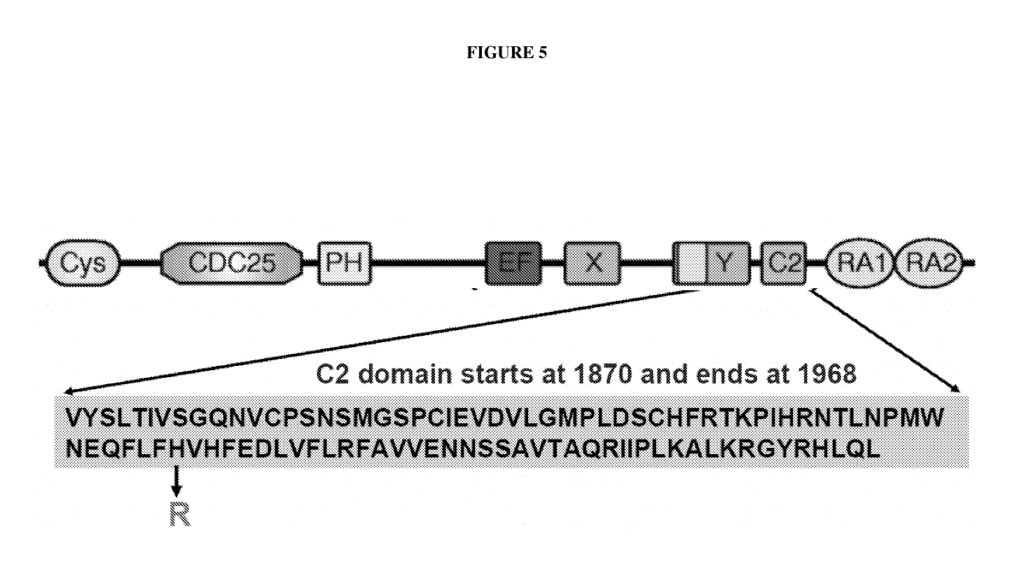

FIG. 6 shows the biological functions of PLCE1. PLCe1 is a member of the PLC family of proteins and cleaves a phospholipid PIP2 into IP3 and diacylglycerol (DAG). IP3 is released as a soluble structure into the cytosol and then binds to IP3 receptors, which opens particular calcium channels in the endoplasmic reticulum, and causes the increase of cytosolic calcium concentration. Calcium and DAG activate protein kinase C, which then phosphorylates cognate substrates, leading to inflammation and cancer.

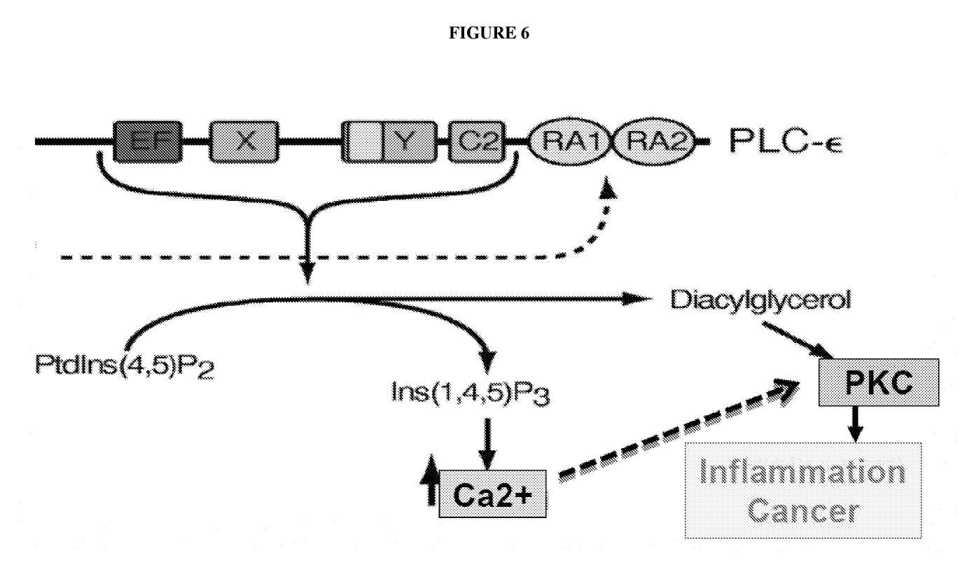

FIG. 7 shows PCLE1 genotypes in human esophageal cancers. 30% (15/50) ESCC cases hasd G allele imbalance (cancer vs. normal).

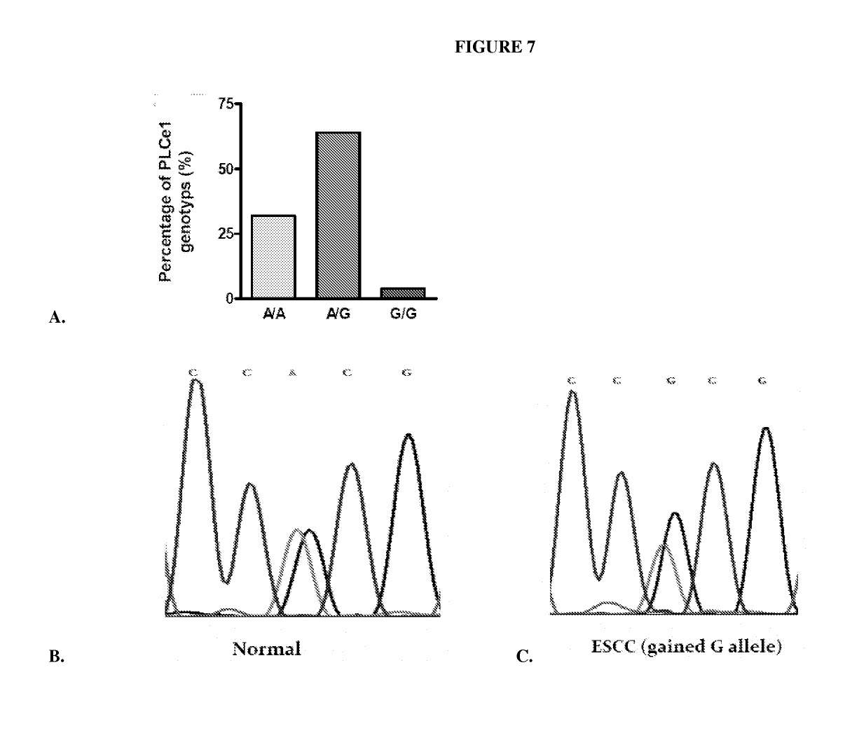

FIG. 8 shows increase of PCLE1 is associated with G allele in SCC as assayed by qRT-PCR and immunohistochemical staining.

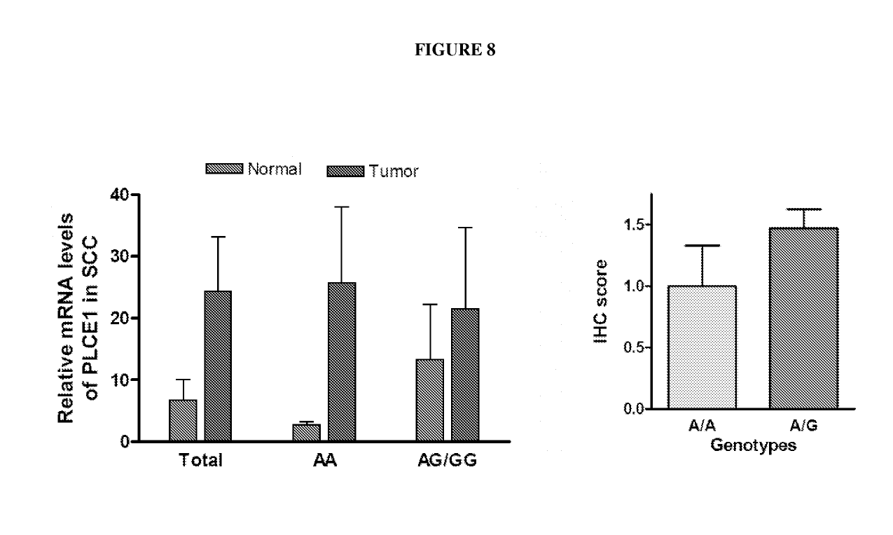

FIG. 9 shows that the G allele is associated with the increase of mRNA in esophageal squamous cell lines (SCC) and adenocarcinoma cell lines (EAC).

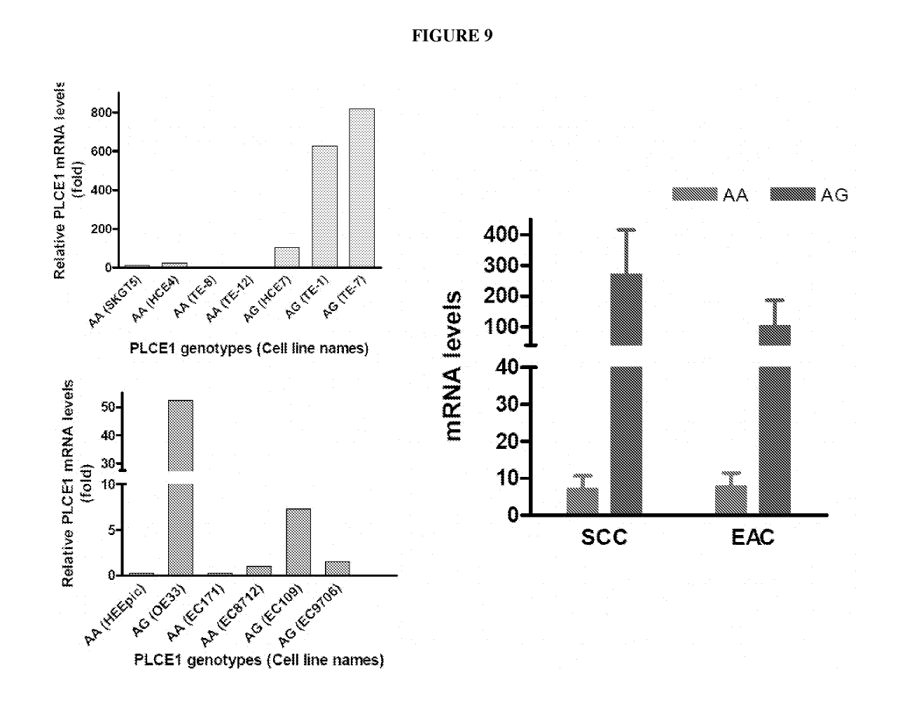

FIG. 10 shows that the G allele is associated with the increase of mRNA and protein levels of PLCE1 in vitro. A correlation of A5780G genotypes with PLCE mRNA, total protein and enzyme activity levels in SCC cell lines. (A) Quantitative determination of PLCE mRNA levels in SCC cell lines (n=13) by qRT-PCR. Bars represent the mean+/−SD for each tumor type. *P<0.05. (B) A representative of immunoblotting showed that PLCE1 protein levels were highter in AG cell lines and were lower in AA cell lines, using specific anti-PLCE antibody. Histograms showed immunoblotting intensities. (C) Mutant (CGC) PLCE1 plasmid showed increased expression of PLCE1 at protein and mRNA levels in the cells with transfection.

FIG. 11 shows that the G allele is associated with increased PLCE enzyme activity. PLCE enzymatic activity was determined by measurement of [3H] inositol triphosphate in esophageal cancer cells with AA and AG alleles. Endogenous PLCE baseline activity (gray bars) was nearly twice as high in the two AG cell lines than in the two AA cell lines (76+/−20 vs. 42+/−7, p<0.05). LPA was used as a ligand to stimulate PLCE activity, and PLCE activites were significantly induced (black bars). Bars represent the mean/−SD for three replicates of experiments performed. *P<0.001, compared to −LPA.

FIG. 12 shows homology binding modeling of PLCE1 C2. HIS 1927 and ion binding sites predicted by the homology models are at 2 ends of the domain.

FIG. 13 shows that the G allele is associated with chronic esophagitis in SCC and non-SCC individuals. The severity of esophagitis (mild, moderate and severe) in SCC (n=58) and non-SCC subjects (n=10614) was correlated with the three PLCE genotypes.

FIG. 14 shows association between PLCE genotypes and esophagitis in non-SCC individuals from high- and low risk areas. The distribution and numbers in each group are provided in FIG. 15. **, P<0.01. The G allele is associated with severe chronic esophagitis in the high-incidence area of esophageal cancer in Northern China.

FIG. 15 shows the association between PLCE1 genotypes and esophagitis in high- and low-risk areas of China.

FIG. 16 shows genetic and expression-level alterations of PLCE in human esophageal cancer tissues. (A) Increased PLCE mRNA levels were determined by RT-PCR in SCC tissues (n=26) compared to adjacent normal control tissues. Bars represent the mean+/−SD of tumor and adjacent normal tissues. **P<0.02. (B) Immunohistochmical staining showed higher PLCE expression scores in SCC than in adjacent normal epithelia. (C) Representative sequencing results showed A5780G allelic imbalance in SCC v. normal esophageal control epithelium. Red arrow indicates a gain of G allele in squamous cell carcinoma.

### DETAILED DESCRIPTION

The inventors have made the surprising discovery that there is an association between disorders of the esophagus, and esophageal-related disorders, and a genetic marker. This genetic marker, or E-marker (for “esophageal marker”), has been identified in PLCE1. The identification of the E-marker in a subject may be useful in predicting disease progression and assessing the subject's response to therapy. In addition, knowledge of this marker associated with a susceptibility to developing disease, or having a disease, may allow one to customize the prevention or treatment in accordance with the subject's genetic profile. A comparison of a subject's PLCE1 profile to a population profile for any particular disorder may permit the selection or design of drugs or other therapeutic regimens that are expected to be safe and efficacious for a particular subject or subject population. Early detection of the E-marker may allow the subject to delay or prevent esophageal-related health complications and/or death.

The ability to target populations expected to show the highest clinical benefit, based on genetic profile, may enable the repositioning of already marketed drugs, the rescue of drug candidates whose clinical development has been discontinued as a result of safety or efficacy limitations, which may be patient sub-group-specific, and/or an accelerated and less costly development of candidate therapeutics.

The methods and materials described below use genetic analysis to determine the presence of the E-marker and reveal whether a subject may have, or be predisposed to, a disorder of the esophagus or esophageal-related disorder.

### 1. Definitions

The terminology used herein is for the purpose of describing particular embodiments only and is not intended to be limiting. As used in the specification and the appended claims, the singular forms “a,” “and” and “the” include plural references unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6,9, and 7.0 are explicitly contemplated.

a. fragment

“Fragment” as used herein may mean a portion of a reference peptide or polypeptide or nucleic acid sequence.

b. identical

“Identical” or “identity” as used herein in the context of two or more polypeptide or nucleotide sequences, may mean that the sequences have a specified percentage of residues or nucleotides that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation.

c. label

“Label” or “detectable label” as used herein may mean a moiety capable of generating a signal that allows the direct or indirect quantitative or relative measurement of a molecule to which it is attached. The label may be a solid such as a microtiter plate, particle, microparticle, or microscope slide; an enzyme; an enzyme substrate; an enzyme inhibitor; coenzyme; enzyme precursor; apoenzyme; fluorescent substance; pigment; chemiluminescent compound; luminescent substance; coloring substance; magnetic substance; or a metal particle such as gold colloid; a radioactive substance such as ¹²⁵I, ¹³¹I, ³²P, ³⁵S, or ¹⁴C; a phosphorylated phenol derivative such as a nitrophenyl phosphate, luciferin derivative, or dioxetane derivative; or the like. The enzyme may be a dehydrogenase; an oxidoreductase such as a reductase or oxidase; a transferase that catalyzes the transfer of functional groups, such as an amino; carboxyl, methyl, acyl, or phosphate group; a hydrolase that may hydrolyzes a bond such as ester, glycoside, ether, or peptide bond; a lyases; an isomerase; or a ligase. The enzyme may also be conjugated to another enzyme.

The enzyme may be detected by enzymatic cycling. For example, when the detectable label is an alkaline phosphatase, a measurement may be made by observing the fluorescence or luminescence generated from a suitable substrate, such as an umbelliferone derivative. The umbelliferone derivative may comprise 4-methyl-umbellipheryl phosphate.

The fluorescent or chemiluminescent label may be a fluorescein isothiocyanate; a rhodamine derivative such as rhodamine B isothiocyanate or tetramethyl rhodamine isothiocyanate; a dancyl chloride (5-(dimethylamino)-1-naphtalenesulfonyl chloride); a dancyl fluoride; a fluorescamine (4-phenylspiro[furan-2(3H); 1ÿ-(3ÿH)-isobenzofuran]-3;3ÿ-dione); a phycobiliprotein such as a phycocyanine or physoerythrin; an acridinium salt; a luminol compound such as lumiferin, luciferase, or aequorin; imidazoles; an oxalic acid ester; a chelate compound of rare earth elements such as europium (Eu), terbium (Tb) or samarium (Sm); or a coumarin derivative such as 7-amino-4-methylcoumarin.

The label may also be a hapten, such as adamantine, fluoroscein isothiocyanate, or carbazole. The hapten may allow the formation of an aggregate when contacted with a multi-valent antibody or (strep)avidin containing moiety. The hapten may also allow easy attachment of a molecule to which it is attached to a solid substrate.

The label may be detected by quantifying the level of a molecule attached to a detectable label, such as by use of electrodes; spectrophotometric measurement of color, light, or absorbance; or visual inspection.

d. linkage Disequilibrium

“Linkage disequilibrium” as used herein may mean the co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage disequilibrium.”

e. Minor Allele Frequency

“Minor allele frequency” as used herein may mean the lowest allele frequency at a locus that is observed in a particular population.

f. Substantially Identical

“Substantially identical,” as used herein may mean that a first and second protein or nucleotide sequence are at least 50%-99% identical over a region of 8-100 or more amino acids nucleotides.

### 2. Method of Diagnosis

Provided herein is a method of diagnosing an esophageal disorder, or a predisposition for an esophageal disorder, in a subject. This diagnosis may be associated with one or more genetic markers and/or corresponding protein markers. The sample may be a nucleic acid and/or protein-containing sample.

The method may call for a qualitative assessment of the presence or absence of the one or more genetic or protein markers in the sample. The method may call for a quantitative assessment of the amount of each of the one or more genetic or protein markers in the sample. These qualitative and quantitative assessments can be made using the herein described methods.

a. Subject

The subject may be a mammal, which may be a human. Prior to diagnosis, the subject may be at risk for cancer because of exposure to one or more risk factors. The one or more risk factors may include, for example, the subject having a family history of cancer, age, smoking tobacco, drinking alcoholic beverages, previous cases of human papilloma virus infections, exposure to radiation, and/or dietary deficiency.

b. Esophageal and Esophageal-Related Disorders

The esophageal disorder may be esophageal cancer and/or esophagitis. The esophagitis may be reflux esophagitis, eosinophilic esophagitis, drug-induced esophagitis, and/or infectious esophagitis. The esophageal-related disorder may be a cancer, other than esophageal cancer, such as gastric cancer, throat cancer, mouth cancer and/or a cancer of the head and/or neck.

c. Sample

The sample may comprise nucleic acid from the subject. The sample may comprise protein from the subject. The nucleic acid may be DNA or RNA. The nucleic acid may be genomic. The sample may be used directly as obtained from the subject or following pretreatment to modify a character of the sample. Pretreatment may include extraction, concentration, inactivation of interfering components, and/or the addition of reagents.

Any cell type, tissue, or bodily fluid may be utilized to obtain a nucleic acid sample. Such cell types, tissues, and fluid may include sections of tissues such as biopsy and autopsy samples, frozen sections taken for histologic purposes, blood, plasma, serum, sputum, stool, tears, mucus, saliva, hair, and skin. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose. Archival tissues, such as those having treatment or outcome history, may also be used. Nucleic acid purification may not be necessary.

d. E-Marker

The E-marker may be a genetic marker. The marker may be a deletion, substitution, insertion, or a polymorphism. The polymorphism may be a single nucleotide polymorphism (SNP). The marker may be in PLCE. PLCE may be found at gene accession number NM_{—}016341. The PLCE may include the nucleic acid at or near the 96066341 region of human chromosome 10q23.

Within a population, a marker may be assigned a minor allele frequency. There may be variations between subject populations. A marker that is common in one geographical or ethnic group may be more rare or uncommon in another. The marker may be overrepresented or underrepresented in a group of subjects. For example, esophageal cancer is one of the most common cancers with a very high mortality rate worldwide and is the fourth highest cause of cancer-related death in China. Epidemiology and etiology studies have suggested the critical roles of environmental and genetic factors in esophageal carcinogenesis in northern China, the area exhibiting the highest rate of incidence in the world. Subjects may be divided into groups on the basis of age, sex/gender, and/or race.

The marker may be detected as a SNP shown in SEQ ID NO:1, wherein nucleotide 401 of SEQ ID NO:1 is a guanine; or a fragment thereof. See Table 1. The fragment may be between 10 and 500 nucleotides, between 50 and 400 nucleotides, between 100 and 300 nucleotides, between 200 and 250 nucleotides, between 10 and 50 nucleotides, between 10 and 20 nucleotides, between 10 and 30 nucleotides, or between 10 and 40 nucleotides in length. The nucleotides of the fragment may be contiguous nucleotides of SEQ ID NO:1, wherein the contiguous sequence contains the guanine at position 401 of SEQ ID NO:1. The fragment may be SEQ ID NO:2, wherein nucleotide 17 is a guanine. The fragment may be SEQ ID NO:14.

The marker may be detected as a SNP shown in SEQ ID NO:3. The marker may be detected as a SNP shown in SEQ ID NO:4, wherein amino acid 6 is an arginine, or SEQ ID NO:7. SEQ ID NO:4 corresponds to the amino acid sequence encoded by SEQ ID NO:2. See Table 1.

TABLE 1

| | dbSNP | |
|---|---|---|
| | accession | |
| Sequence | no. | SEQ ID NO. |
| ATGGTCTCAA TCTCCTGACC TCGTGATCTG | rs2274223 | 1 |
| CTCACCTCAG CCTCCCAAAG TGCTGGGATT | | |
| ACAGGCATAA GCCACCGCGC CCAGCCCTAC | | |
| AATCACTTAC TTTTTAAACA GTTTTATTCA | | |
| TCATTCACTT TGTCCATTCC AGTGTTCTTG | | |
| GGATTCCTTT GCAGAGGGAA GCAGTGAGGT | | |
| GCAGAGGTTG TCTTTCTTTT TTATCCTCGG | | |
| TGACTTTGAT CCCTTTTGTC TCCCTCACCC | | |
| TAGATTGTCT CTGGTCAGAA TGTGTGCCCC | | |
| AGTAATAGCA TGGGAAGCCC GTGCATTGAA | | |
| GTCGACGTCC TGGGCATGCC TCTGGACAGC | | |
| TGCCATTTCC GCACAAAGCC CATCCATCGA | | |
| AACACCCTGA ACCCCATGTG GAACGAGCAG | | |
| TTTCTGTTCC | | |
| G | | |
| CGTTCACTTC GAAGATCTTG TATTTCTTCG | | |
| TTTTGCAGTT GTGGAAAACA ATAGTTCAGC | | |
| GGTAACTGCT CAGAGAATCA TTCCACTGAA | | |
| AGCTTTAAAA CGAGGTAGAA TAAAATTGTC | | |
| CAAATGTTAA TAATTGTTGT AGCTAGGTGA | | |
| TGGATGCCAG AATTTCCTTA TACTCTTCTC | | |
| TCTTTTCTGT TTGACATTTT CCTATAAAGA | | |
| AGTTGAATTA AGAAGCAAAA GGAAATACAA | | |
| ATTGGAGCAC TTTTGAAGCA CATCTCAAAA | | |
| GAAGTTTTTC TTTTACATTT TTTAAGAATC | | |
| TGAGGTATTA ACAATATCCC TTGTAAAGTA | | |
| CAGCCTCACC CATGCTTGCT GATGTCAGGA | | |
| GCAGTGGTTG TTGCTACCCA CGGCACTGCT | | |
| GGCAGTTTGA | | |
| GAGCAG TTTCTGTTCC | Fragment of | 2 |
| G | SEQ ID | |
| CGTTCACTTC GAAGAT | NO: 1 | |
| IVSGQNVCPSNSMGSPCIEVDVLGMPLDSCHFRTKPI | Amino acid | 3 |
| HRNTLNPMWNEQFLFRVHFEDLVFLRFAVVENNSSAV | sequence | |
| TAQRIIPLKALKR | encoded by | |
| | fragment of | |
| | SEQ ID | |
| | NO: 1 (See | |
| | SEQ ID | |
| | NO: 14). | |
| EQFLFRVHFED | Amino acid | 4 |
| | sequence | |
| | encoded by | |
| | fragment of | |
| | SEQ ID | |
| | NO: 1 (See | |
| | SEQ ID | |
| | NO: 2). | |
| GAGCAG TTTCTGTTCC | Wild type | 5 |
| A | nucleotide | |
| CGTTCACTTC GAAGAT | fragment of | |
| | PLCE. | |
| EQFLFHVHFED | Wild type | 6 |
| | peptide | |
| | fragment of | |
| | PLCE | |
| | encoded by | |
| | SEQ ID | |
| | NO: 5. | |
| VYSLTIVSGQNVCPSNSMGSPCIEVDVLGMPLDSCHF | Amino acid | 7 |
| RTKPIHRNTLNPMWNEQFLFRVHFEDLVFLRFAVVEN | sequence | |
| NSSAVTAQRIIPLKALKRGYRHLQL | encoded by | |
| | fragment of | |
| | SEQ ID | |
| | NO: 1. | |
| ATTGTCTCTGGTCAGAATGTGTGCCCCAGTAATAGCA | Nucleotide | 14 |
| TGGGAAGCCCGTGCATTGAAGTCGACGTCCTGGGCAT | sequence | |
| GCCTCTGGACAGCTGCCATTTCCGCACAAAGCCCATC | encoding | |
| CATCGAAACACCCTGAACCCCATGTGGAACGAGCAGT | SEQ ID | |
| TTCTGTTCCGCGTTCACTTCGAAGATCTTGTATTTCT | NO: 3. | |
| TCGTTTTGCAGTTGTGGAAAACAATAGTTCAGCGGTA | | |
| ACTGCTCAGAGAATCATTCCACTGAAAGCTTTAAAAC | | |
| GA | | |

In addition to detecting, or measuring the amount of one or more of the markers set forth in Table 1, detecting the presence or absence of other markers, or measuring the amounts thereof, useful for diagnosing cancer may also be provided. For example, the presence of trefoil factor 2 (TFF2) immunoreactive cells is associated with gastric cancers. See See Schmidt, P. H., Lee, J. R., Joshi, V., Playford, R. J., Poulsom, R., Wright, N. A., and Goldenring, J. R. 1999. Identification of a metaplastic cell lineage associated with human gastric adenocarcinoma. Lab. Invest. 79:639-646. Further, certain ratios of pepsinogen I/II have been associated with certain upper GI cancers. Lee e.g., Oishi Y, Kiyohara Y, Kubo M, Tanaka K, Tanizaki Y, Ninomiya T, Doi Y, Shikata K, Yonemoto K, Shirota T, Matsumoto T. Lida M. 2006. The serum pepsinogen test as a predictor of gastric cancer: the Hisayama study, Am J Epidemiol. 163(7):629-37; and Dinis-Ribeiro M, da Costa-Pereira A, Lopes C, Barbosa J, Guilherme M, Moreira-Dias L, Lomba-Viana H, Silva R, Abreu N, Lomba-Viana R. 2004. Validity of serum pepsinogen I/II ratio for the diagnosis of gastric epithelial dysplasia and intestinal metaplasia during the follow-up of subjects at risk for intestinal-type gastric adenocarcinoma. Neoplasia 6(5):449-56. As such, TFF2 and/or pepsinogen I and pepsinogen II (to determine pepsinogen II/I ratios) can be additionally measured to create a diagnostic matrix for identification of those at risk for or presently suffering from upper GI cancer. One or more other markers may also be selected from HE4, LGALS3, IL1RN, TRIP133, FIGNL1, CRIP1, S100A4, EXOSC8, EXPI, BRRN1, NELF, EREG, TMEM40 and TMEM109.

The analysis of markers can be carried out separately or simultaneously with additional other markers within one test patient sample. For example, several markers can be combined into one test for efficient processing of a multiple of samples and for potentially providing greater diagnostic and/or prognostic accuracy. In addition, multiple samples (for example, at successive time points) from the same test patient may be analyzed. Such testing of serial samples can allow the identification of changes in marker levels over time. Increases or decreases in marker levels, as well as the absence of change in marker levels, can provide useful information about the cancer status that includes identifying the approximate time from onset of the cancer, the presence and amount of salvageable tissue, the appropriateness of drug therapies, the effectiveness of various therapies, and identification of the subjects outcome, including risk of future cancer-related events, such as metastasis.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in timely fashion, for example, in ambulatory transport or in emergency room settings.

e. Detection—Polynucleotides

The E-marker may be detected in a sample derived from the patient. Many methods are available for detecting a marker in a subject and may be used in conjunction with the herein described methods. These methods include large-scale SNP genotyping, exonuclease-resistant nucleotide detection, solution-based methods, genetic bit analyses, primer guided nucleotide incorporation, allele specific hybridization, and other techniques. Any method of detecting a marker may use a labeled oligonucleotide.

(1) Large Scale SNP Genotyping

Large scale SNP genotyping may include any of dynamic allele-specific hybridization (DASH), microplate array diagonal gel electrophoresis (MADGE), pyrosequencing, ologonucleotide-specific ligation, or various DNA “chip” technologies such as Affymetrix SNP chips. These methods may require amplification of the target genetic region. Amplification may be accomplished via polymerase chain reaction (PCR).

(2) Exonuclease-Resistant Nucleotide

PI-markers may be detected using a specialized exonuclease-resistant nucleotide, as described in U.S. Pat. No. 4,656,127, which is incorporated herein by reference. A primer complementary to the allelic sequence immediately 3′ to the polymorphic site may be permitted to hybridize to a target molecule obtained from the subject. If the polymorphic site on the target molecule contains a nucleotide that is complementary to the particular exonuclease-resistant nucleotide derivative present, then that derivative may be incorporated onto the end of the hybridized primer. Such incorporation may render the primer resistant to exonuclease, and thereby permit its detection. Since the identity of the exonuclease-resistant derivative of the sample may be known, a finding that the primer has become resistant to exonuclease reveals that the nucleotide present in the polymorphic site of the target molecule was complementary to that of the nucleotide derivative used in the reaction. This method may not require the determination of large amounts of extraneous sequence data.

(3) Solution-Based Method

A solution-based method may be used to determine the identity of a PI-marker, as described in PCT Application No. WO91/02087, which is herein incorporated by reference. A primer may be employed that is complementary to allelic sequences immediately 3′ to a polymorphic site. The method may determine the identity of the nucleotide of that site using labeled dideoxynucleotide derivatives that, if complementary to the nucleotide of the polymorphic site, will become incorporated onto the terminus of the primer.

(4) Genetic Bit Analysis

Genetic bit analysis may use mixtures of labeled terminators and a primer that is complementary to the sequence 3′ to a polymorphic site. A labeled terminator may be incorporated, wherein it is determined by and complementary to, the nucleotide present in the polymorphic site of the target molecule being evaluated. The primer or the target molecule may be immobilized to a solid phase.

(5) Primer-Guided Nucleotide Incorporation

A primer-guided nucleotide incorporation procedure may be used to assay for a PI-marker in a nucleic acid, as described in Nyren, P. et al., Anal. Biochem. 208:171-175 (1993), which is herein incorporated by reference. Such a procedure may rely on the incorporation of labeled deoxynucleotides to discriminate between bases at a polymorphic site. In such a format, since the signal is proportional to the number of deoxynucleotides incorporated, polymorphisms that occur in runs of the same nucleotide may result in signals that are proportional to the length of the run.

(6) Allele Specific Hybridization

Allele specific hybridization may be used to detect a PI-marker. This method may use a probe capable of hybridizing to a target allele. The probe may be labeled. A probe may be an oligonucleotide. The target allele may have between 3 and 50 nucleotides around the marker. The target allele may have between 5 and 50, between 10 and 40, between 15 and 40, or between 20 and 30 nucleotides around the marker. A probe may be attached to a solid phase support, e.g., a chip. Oligonucleotides may be bound to a solid support by a variety of processes, including lithography. A chip may comprise more than one allelic variant of a target region of a nucleic acid, e.g., allelic variants of two or more polymorphic regions of a gene.

(7) Other Techniques

Examples of other techniques for detecting alleles include selective oligonucleotide hybridization, selective amplification, or selective primer extension. Oligonucleotide primers may be prepared in which the known mutation or nucleotide difference is placed centrally and then hybridized to target DNA under conditions which permit hybridization if a perfect match is found. Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labeled target DNA.

Allele specific amplification technology that depends on selective PCR amplification may be used in conjunction with the instant invention. Oligonucleotides used as primers for specific amplification may carry the mutation or polymorphic region of interest in the center of the molecule. Amplification may then depend on differential hybridization, as described in Gibbs et al. (1989) Nucleic Acids Res. 17:2437-2448), which is herein incorporated by reference, or at the extreme 3′ end of one primer where, under appropriate conditions, mismatch can prevent, or reduce polymerase extension.

Direct DNA sequencing, either manual sequencing or automated fluorescent sequencing may detect sequence variation. Another approach is the single-stranded conformation polymorphism assay (SSCP), as described in Orita M, et al. (1989) Proc. Natl. Acad. Sci. USA 86:2766-2770, which is incorporated herein by reference. The fragments that have shifted mobility on SSCP gels may be sequenced to determine the exact nature of the DNA sequence variation. Other approaches based on the detection of mismatches between the two complementary DNA strands include clamped denaturing gel electrophoresis (CDGE), as described in Sheffield V C, et al. (1991) Am. J Hum. Genet. 49:699-706, which is incorporated herein by reference; heteroduplex analysis (HA), as described in White M B, et al. (1992) Genomics 12:301-306, which is incorporated herein by reference; and chemical mismatch cleavage (CMC) as described in Grompe M, et al., (1989) Proc. Natl. Acad. Sci. USA 86:5855-5892, which is herein incorporated by reference. A review of currently available methods of detecting DNA sequence variation can be found in a review by Grompe (1993), which is incorporated herein by reference. Grompe M (1993) Nature Genetics 5:111-117. Once a mutation is known, an allele specific detection approach such as allele specific oligonucleotide (ASO) hybridization can be utilized to rapidly screen large numbers of other samples for that same mutation. Such a technique can utilize probes that may be labeled with gold nanoparticles to yield a visual color result as described in Elghanian R, et al. (1997) Science 277:1078-1081, which is herein incorporated by reference.

A rapid preliminary analysis to detect polymorphisms in DNA sequences can be performed by looking at a series of Southern blots of DNA cut with one or more restriction enzymes, preferably with a large number of restriction enzymes.

f. Detection—Polypeptide

The E-marker may also be detected by a corresponding E-marker polypeptide. With regard to determining the presence and/or amount of E-marker polypeptide in a sample, mass spectrometry and/or immunoassay devices and methods may be used. Other methods include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety.

(1) Immunoassay

E-marker peptides may be analyzed using an immunoassay. The presence or amount of a E-marker can be determined using antibodies or fragments thereof specific for each biomarker polypeptide, or fragment thereof, and detecting specific binding. For example, the antibody, or fragment thereof, may specifically bind to a polypeptide comprising SEQ ID NO:3, SEQ ID NO:4, SEQ ID NO:6, SEQ ID NO:7, or a fragment thereof. The antibody, or fragment thereof, may specifically bind to a polypeptide consisting of SEQ ID NO:3, SEQ ID NO:4, SEQ ID NO:6, SEQ ID NO:7, or a fragment thereof. The antibody may be polyclonal or monoclonal.

Any immunoassay may be utilized. The immunoassay may be an enzyme-linked immunoassay (ELISA), radioimmunoassay (RIA) or a competitive binding assay, for example. Specific immunological binding of the antibody to the marker can be detected via direct labels, such as fluorescent or luminescent tags, metals and radionuclides attached to the antibody or via indirect labels, such as alkaline phosphatase or horseradish peroxidase.

The use of immobilized antibodies or fragments thereof may be used in the immunoassay. The antibodies may be immobilized onto a variety of solid supports, such as magnetic or chromatographic matrix particles, the surface of an assay plate (such as microtiter wells), pieces of a solid substrate material, and the like. An assay strip can be prepared by coating the antibody or plurality of antibodies in an array on a solid support. This strip can then be dipped into the test biological sample and then processed quickly through washes and detection steps to generate a measurable signal, such as a colored spot.

(a) Antibodies

Monoclonal and/or polyclonal antibodies may be used. A monoclonal antibody refers to an antibody that is derived from a single clone, including any eukaryotic, prokaryotic, or phage clone. The monoclonal antibody may comprise, or consist of, two proteins, i.e., heavy and light chains. The monoclonal antibody can be prepared using one of a wide variety of techniques known in the art including the use of hybridoma, recombinant, and phage display technologies, or a combination thereof.

Anti-SEQ ID NO:3, 4, 6, or 7 monoclonal antibodies may be prepared using any known methodology, including the seminal hybridoma methods, such as those described by Kohler and Milstein (1975), Nature. 256:495. Monoclonal antibodies may be prepared against any amino acid sequence provided herein, including the wild type PLCE protein. In a hybridoma method, a mouse, hamster, or other appropriate host animal is immunized with an immunizing agent to elicit lymphocytes that produce or are capable of producing antibodies that will specifically bind to the immunizing agent. Alternatively, the lymphocytes may be immunized in vitro.

The immunizing agent will typically include at least a portion of the desired polypeptide or a fusion protein thereof. For example, synthetic polypeptide or recombinant polypeptide comprising any PLCE epitopes may be used as an immunizing agent. Exemplary epitopes include, but are not limited to, SEQ ID NOs:3, 4, 6, and 7. A fusion protein may be made by fusing a polypeptide to a carrier protein, for example, keyhole limpet hemocyanin (KLH, EMD Biosciences, San Diego, Calif.), BSA (EMD Biosciences, San Diego, Calif.), or ovalbumin (Pierce, Rockford, Ill.). The immunizing agent may be administered to a mammal with or without adjuvant according to any of a variety of standard methods. The immunizing agent may be administered only once, but is preferably administered more than once according to standard boosting schedules.

Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell population which is screened for species having appropriate specificity and affinity to epitopes on the N-terminal portion of galectin-3 (Goding, (1986) Monoclonal Antibodies: Principles and Practice, Academic Press, pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Immortalized cell lines that fuse efficiently, support stable high level expression of antibody by the selected antibody-producing cells, and are sensitive to a medium such as HAT medium may be used. Immortalized cell lines that are murine myeloma lines, which can be obtained, for instance, from the Salk Institute Cell Distribution Center, San Diego, Calif. and the American Type Culture Collection, Manassas, Va. may be used. Human myeloma and mouse-human heteromyeloma cell lines may be used and have been described for the production of human monoclonal antibodies (Kozbor, J. (1984) Immunol., 133:3001; Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the desired polypeptide, e.g., by screening with a labeled desired polypeptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard (1980), Anal. Biochem., 107:220. Various analysis protocols to determine binding specificity are available commercially as kits or as a service.

Monoclonal antibodies also may be made by recombinant DNA methods, such as those described in U.S. Pat. No. 4,816,567. DNA encoding suitable monoclonal antibodies can be isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al., (1984) Proc. Natl. Acad. Sci. USA, 81:6851) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

Antibodies can also be produced using phage display libraries (Hoogenboom and Winter (1991), J. Mol. Biol. 227:381; Marks et al. (1991), J. Mol. Biol., 222:581). The techniques of Cole et al. and Boerner et al. are also available for the preparation of monoclonal antibodies (Cole et al. (1985), Monoclonal Antibodies and Cancer Therapy, Alan R. Liss, p. 77 and Boerner et al. (1991), J. Immunol., 147(1):86-95). Similarly, antibodies can be made by introducing of immunoglobulin loci into transgenic animals, e.g., mice in which the endogenous immunoglobulin genes have been partially or completely inactivated.

The antibodies may also be affinity matured using known selection and/or mutagenesis methods as described above. Affinity matured antibodies may have an affinity which is five times, 10 times, 20 or 30 times greater than the starting antibody from which the matured antibody is prepared. A capture binding moiety may be the anti-SEQ ID NO:3, 4, 6 or 7 monoclonal antibody. A labeled detection binding moiety may be a second anti-SEQ ID NO:3, 4, 6 or 7 monoclonal antibody.

Other binding moieties may be used with the methods and kits of the present invention. Examples of binding moieties include, but are not limited to, proteins, peptide aptamers, avimers, Adnectins and AFFIBODY® ligands; nucleic acids, such as DNA and RNA (including nucleotide aptamers), and lipids, such as membrane lipids.

(2) Mass Spectrometry

Mass spectrometry (MS) analysis may be used alone or in combination with other methods. Other methods include immunoassays and those described above to detect specific polynucleotides. The mass spectrometry method may be used to determine the presence and/or quantity of one or more biomarkers. MS analysis may comprise matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as liquid chromatography (LC) ESI-MS. Mass analysis can be accomplished using commercially-available spectrometers. Methods for utilizing MS analysis, including MALDI-TOF MS and ESI-MS, to detect the presence and quantity of biomarker peptides in biological samples may be used. See, for example, U.S. Pat. Nos. 6,925,389; 6,989,100; and 6,890,763 for guidance, each of which is incorporated herein by reference.

g. Amplification of E-Marker

Any method of detection may incorporate a step of amplifying the PI-marker. A PI-marker may be amplified and then detected. Nucleic acid amplification techniques may include cloning, polymerase chain reaction (PCR), PCR of specific alleles (ASA), ligase chain reaction (LCR), nested polymerase chain reaction, self-sustained sequence replication, transcriptional amplification system, and Q-Beta Replicase, as described in Kwoh, D. Y. et al., 1988, Bio/Technology 6:1197, which is incorporated herein by reference.

Amplification products may be assayed by size analysis, restriction digestion followed by size analysis, detecting specific tagged oligonucleotide oligonucleotide primers in reaction products, allele-specific oligonucleotide (ASO) hybridization, allele specific 5′ exonuclease detection, sequencing, and/or hybridization.

PCR-based detection means may include amplification of a plurality of markers simultaneously. PCR primers may be selected to generate PCR products that do not overlap in size and may be analyzed simultaneously. Alternatively, one may amplify different markers with primers that are differentially labeled. Each marker may then be differentially detected. Hybridization-based detection means may allow the differential detection of multiple PCR products in a sample.

Nucleic acid primers and/or oligonucleotides may be used in conjunction with any of the herein described methods and/or kits. See below Examples. The following oligonucleotides or primers may be present in the herein described kits and/or used in the herein described methods:

TABLE 2

| SNP | Primer #1 | Primer #2 |
|---|---|---|
| PLCE | TGTTCTTGGGATTCCTTTGC | TGCTTCTTAATTCAACTTCTTTATAGG |
| exon 26 | (SEQ ID NO: 8) | (SEQ ID NO: 9) |
| PLCE C2 | TGTGGAACGAGCAGTTTCTG | ATCGAAGAGGCTGACATGGT |
| domain | (SEQ ID NO: 10) | (SEQ ID NO: 11) |
| PLCE | TGTTCTTGGGATTCCTTTGC | TGCTTCTTAATTCAACTTCTTTATAGG |
| exon 26 | (SEQ ID NO: 12) | (SEQ ID NO: 13) |

The probe or oligonucleotide may be a fragment of any one of SEQ NO:1 or SEQ ID NO:2, wherein the fragment comprises a contiguous nucleic acid sequence and the corresponding SNP of SEQ ID NO:1 or SEQ ID NO:2. The fragment may be between 10 and 500 nucleotides, between 50 and 400 nucleotides, between 100 and 300 nucleotides, between 200 and 250 nucleotides, between 10 and 50 nucleotides, between 10 and 20 nucleotides, between 10 and 30 nucleotides, or between 10 and 40 nucleotides in length.

h. Control

It may be desirable to include a control sample that is analyzed concurrently with the sample from the subject described above. The results obtained from the subject sample can be compared to the results obtained from the control sample. Standard curves may be provided, with which assay results for the biological sample may be compared. Such satandard curves present levels of marker as a function of assay units, i.e., fluorescent signal intensity, if a fluorescent lable is used. Using samples taken from multiple donors, standard curves can be provided for control levels of the one or more biomarkers in normal tissue, as well as for “at-risk” levels of the one or more biomarkers in tissue taken from donors with metaplasia or from donors with upper GI cancer.

### 3. Method of Treatment

In any patient that carries the E-marker, an assessment may be made as to whether the subject has a disorder of the esophagus, is not at risk of a disorder of the esophagus, having a low risk of a disorder of the esophagus, or having a high risk of a disorder of the esophagus. For example, an assessment may be made as to whether the subject has cancer, is not at risk of cancer, having a low risk of cancer, or having a high risk of cancer. The assessment may indicate an appropriate course of preventative or maintenance therapy. For example, a subject may be diagnosed as having a GI cancer if, when compared to a control, there is a measurable difference in the amount of the at least one marker in the sample. When no marker is identified in the sample, the subject can be identified as not having upper GI cancer, not being at risk for the cancer, or as having a low risk of the cancer. In this regard, subjects having the cancer or risk thereof can be differentiated from subjects having low to substantially no cancer or risk therof. Thos subjects having a risk of developing an upper GI cancer can be placed on a more intensive and/or regular screening schedule, including upper endoscopic surveillance. On the other hand, those subjects having low to substantially no risk may avoid being subjected to an endoscopy, until such time as a future screening indicates that a risk of upper GI cancer has appeared in those subjects.

Therapy may be administered in different clinical settings during the life of a subject: (1) during early stages of an esophageal disorder, a subject may receive anti-inflammatories or antibiotics to delay onset of chronic bacterial colonization; (2) after a subject has been colonized with one or more bacterial pathogens, wherein antibiotics may be administered to slow any decline in pulmonary function and reduce frequency and morbidity of pulmonary exacerbations; and/or (3) during periodic exacerbations in pulmonary symptoms, wherein intensive antibiotic regimens may be administered to relieve symptomatology and restore normal function to baseline values.

a. Predictive Treatment

Provided herein is a method of treating a subject having a marker as described herein. The subject may be recommended to eat a balanced diet, avoid excess sun exposure, wear sun block and/or a hat when outdoors in the sunlight. The subject may be discouraged from smoking cigarettes or cigars and should not be exposed to second hand smoke. The subject may be discouraged from eating spicy foods such as those with pepper, chili powder, curry and nutmeg. The subject may be discouraged from eating hard foods such as nuts, crackers, and raw vegetables. The subject may be discouraged from eating acidic foods and beverages such as tomatoes, oranges, grapefruits and their juices. The subject may be prescribed a regimen of soft foods. Anti-inflammatories and/or ntibiotics may be administered to the subject to prevent or delay onset of the esophageal disorder.

The treatment of a subject with a particular therapeutic may be monitored by determining protein, mRNA, and/or transcriptional level of a gene. The gene may be in the phospholipase C epsilon gene. Depending on the level detected, the therapeutic regimen may be maintained or adjusted. The effectiveness of treating a subject with an agent may comprise (1) obtaining a preadministration sample from a subject prior to administration of the agent; (2) detecting the level or amount of a protein, RNA or DNA in the preadministration sample; (3) obtaining one or more post-administration samples from the subject; (4) detecting the level of expression or activity of the protein, RNA or DNA in the postadministration sample; (5) comparing the level of expression or activity of the protein, RNA or DNA in the preadministration sample with the corresponding protein, RNA, or DNA in the postadministration sample, respectively; and (6) altering the administration of the agent to the subject accordingly.

Cells of a subject may be obtained before and after administration of a therapeutic to detect the level of expression of genes other than the gene of interest to verify that the therapeutic does not increase or decrease the expression of genes that could be deleterious. Verification may be accomplished by transcriptional profiling. mRNA from cells exposed in vivo to a therapeutic and mRNA from the same type of cells that were not exposed to the therapeutic may be reverse transcribed and hybridized to a chip containing DNA from many genes. The expression of genes in the treated cells may be compared against cells not treated with the therapeutic.

b. Maintenance Therapy

Appropriate antibiotic therapy and/or anti-inflammatory therapy may be essential steps in the management of an esophageal disorder. Antibiotic selection for any given subject in any given setting may be based on periodic isolation and identification of pathogens from respiratory secretions, for example, and a review of the antimicrobial susceptibility profile for those pathogens. Antibiotics may be used for outpatient management of the disorder and/or for the treatment of bacteria associated with the disorder.

c. Antibiotics, Anti-Inflammatories, and Proton Pump Inhibitors

An antibiotic may be selected from the following: an aminoglycoside, amoxicillin, levofloxacin, dicloxacillin, cephalexin, amoxicillin/clavulanate, erythromycin, clarithromycin, azithromycin, clindamycin, cefuroxime axetil, cefprozil, cefixime, cefpodoxime proxetil, loracarbef, ciprofloxacin, tobramycin, colistin, trimethoprim/sulfamethoxazole, doxycycline, minocycline, cefazolin, nafcillin, vancomycin, β-lactam, ceftazidime, ticarcillin, piperacillin, imipenem, meropenem, aztreonam, an aminoglycoside, amikacin, merpenem, ceftazidime, chloramphenicol, ticarcillin/clavulanate, aztreonam, imipenem, a polypeptide antibiotic, and/or meropenem. The polypeptide antibiotic may be of the polymyxin class of antibiotics. A broad range antibiotic may be used in the regimen. A broad range antibiotic may include levofloxacin or amoxycillin.

Any anti-inflammatory agent may be used. The anti-inflammatory may be ibuprofen, an oral sterioid, and/or an inhaled steroid.

A proton pump inhibitor, which may block acid production in the stomach and allow time for esophageal tissue to heal, may be selected from the group consisting of omeprazole, esomeprazole, and/or lansoprazole.

The antibiotic, anti-inflammatory, or proton pump inhibitor may be formulated for administration by injection, inhalation or insufflation through the nose or mouth, or oral, buccal, parenteral, or rectal administration. The antibiotic or anti-inflammatory may be formulated for parenteral administration by injection, e.g., by bolus injection or continuous infusion. Formulations for injection may be presented in unit dosage form, e.g., in ampules or in multi-dose containers, with an added preservative. The antibiotic or anti-inflammatory may take such a form as a suspension, solution, or emulsion in oily or aqueous vehicles, and may contain formulating agents such as suspending, stabilizing and/or dispersing agents. Antibiotic or anti-inflammatory preparations for oral administration may be suitably formulated to give controlled release of the antibiotic. For buccal administration, the antibiotic may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant. In the case of a pressurized aerosol, the dosage unit may be determined by providing a valve to deliver a metered amount.

An effective dose of the antibiotic may be based upon a culture determination of the bacterial type causing the infection. In addition, an antimicrobial susceptibility report may indicate which families of antibiotic drugs are useful for the particular bacteria recovered from the infection. If the cause of the infection is unclear, but suspected to be due to bacteria, a broad-spectrum antibiotic may be prescribed for controlling a wide variety of bacterial types. In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day. Therapeutically effective amounts of an antibiotic may range from approximately 0.05 mg to 10 g per kilogram body weight of the subject per day.

### 4. Method of Monitoring the Esophageal Disorder

Also provided herein is a method of monitoring a subject for an esophageal disorder. The subject may have been determined to have a predisposition for an esophageal disorder. It may be desirable to measure the effects of treatment on the disorder by treating the subject using a method comprising monitoring the disorder. Monitoring for cancer or inflammation of the esophagus, or progression of cancer or inflammation of the esophagus, for example, may include any imaging techniques, inflammatory markers, identification of serological markers, and any of several general signs such as persistent cough or blood-tinged saliva, a change in bowel habits, blood in the stool, and/or unexplained anemia.

### 5. Kit

Provided herein is a kit, which may be used for diagnosing, monitoring, or treating an esophageal disorder or esophageal-related disorder. The kit may comprise probes, for example antibodies, selective for marker polypeptides or nucleic acid hybridization probes that can selectively bind mRNA (or cDNA amplified therefrom), for example, having specificity for one or more markers disclosed herein. The probes may be bound to a substrate. Such a kit can comprise devices and reagents for the analysis of at least one test sample. The kit can further comprise instructions for using the kit and conducting the analysis. Optionally, the kits can contain one or more reagents or devices for converting a marker level to a diagnosis or prognosis of the subject.

The kit may comprise a sample collecting means; for example, a nucleic acid sample collecting means. The kit may also comprise a means for determining a marker in a PLCE gene sequence or peptide sequence, a nucleic acid or peptide for use as a positive control, and/or a nucleic acids or peptide sampling means. The nucleic acid or protein sampling means may include substrates, such as filter paper, nucleic acid purification reagents, such as reaction buffer, polymerase, and dNTPs. Marker detection means may also be included in the kit. Such means may include antibodies, specific restriction enzymes, marker specific oligonucleotides, and degenerate oligonucleotide primers for PCR. The positive control may be used for nucleotide or amino acid sequence comparison.

The kit may also comprise one or more containers, such as vials or bottles, with each container containing a separate reagent. The kit may further comprise written instructions, which may describe how to perform or interpret an assay or method described herein.

The present invention has multiple aspects, illustrated by the following non-limiting examples.

### EXAMPLES

### Example 1

### PLCE1 SNP Associated with Esophageal Carcinogenesis

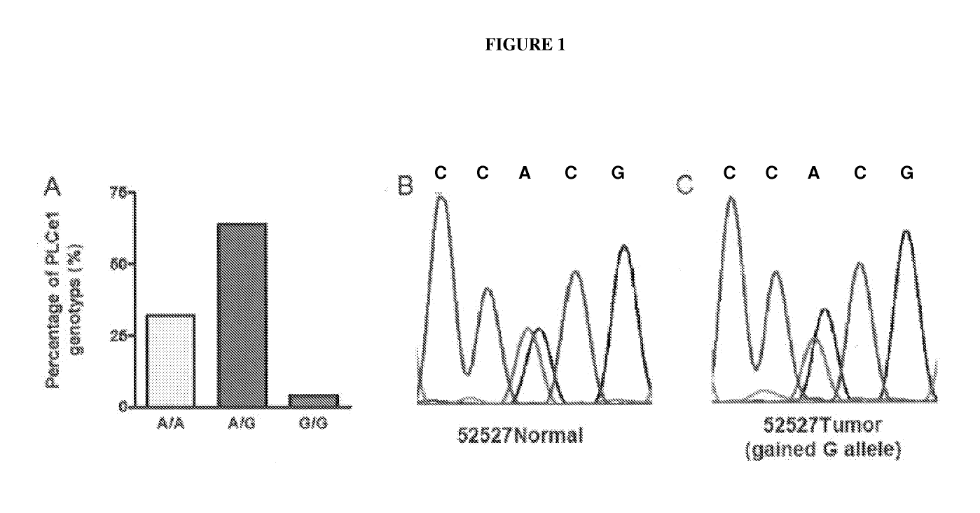

Genome-wide association study (GWAS) is believed to be a powerful tool to identify susceptible loci associating with diseases. A GWAS was performed on esophageal squamous cell carcinoma (SCC) by genotyping 1,077 cases and 1,733 cases and 1,733 controls in Chinese at the first stage. 18 SNPs were selected for replication in an additional 7,673 SCC cases and 11,013 controls of Chinese at stage 2. Two novel susceptibility loci for SCC were identified: one is rs2274223 (A to G) located in the PLCE1 gene on chromosome 10q23 (P=1.18E-55, OR=1.43), and another one, rs13042395 (C to T) located in the C20orf54 gene on 20p13 (P=1.23E-11, OR=0.86). These results strongly suggest that the SNPs, particularly the rs2274223 at the PCLE1 gene is an esophageal cancer susceptible genetic variant. See FIG. 1.

There are 32 exons in human PLCE1 gene, and the rs2274223 is located in the exon 26, causing missense mutation (CAC to CGC corresponding to histidine to arginine). The amino acid sequence for exon 26 having the arginine is shown herein as SEQ ID NO:3. To determine whether there is a gain of G allele (or G allele imbalance), we extracted DNA from 50 paired of esophageal cancer and adjacent normal esophageal mucosa of Chinese population, ran a PCR-based sequencing and evaluated G allele imbalance. Three genotypes of PLCE1 (AA, AG and GG) were identified (FIG. 1A) and the majority (60%) was AG (heterozygous), which was higher than that (about 40%) generated from the genome DNA from white blood cells in GWAS. This might be the result of increased G allele mutation in cancers. As shown in FIG. 1B and 1C, some of the AG cancers showed gain of G allele (allele imbalance) compared to the normal control.

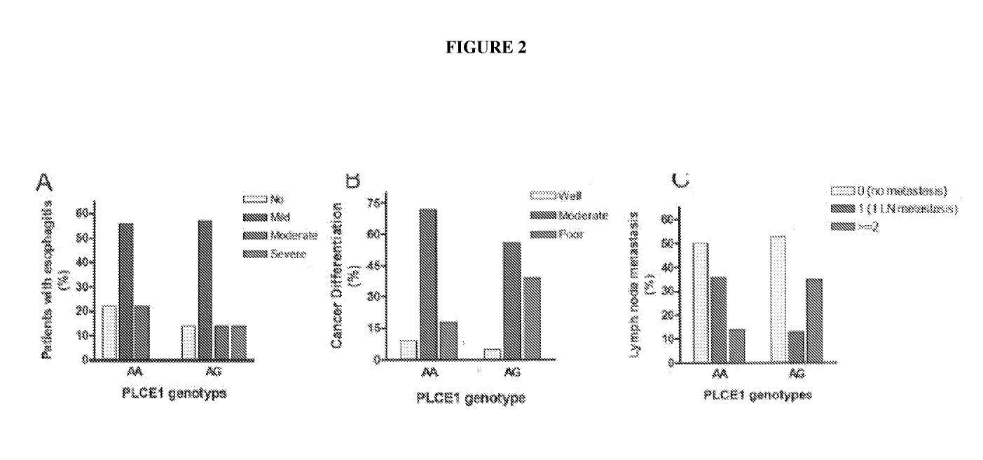

To determine biological functions of PLCE1 in esophageal carcinogenesis, the G allele in 30 Chinese SCC and found that the presence of the G allele was associated with an increase of esophagitis (FIG. 2A), poor cancer cell differentiation (FIG. 2B), and lymph node metastasis (FIG. 2C). The presence of the G allele and gain of G allele would be well associated with more histopathological features (e.g. stages) and prognosis.

### Example 2

### G Allele is Associated with Increased PLCE1 Gene Expression

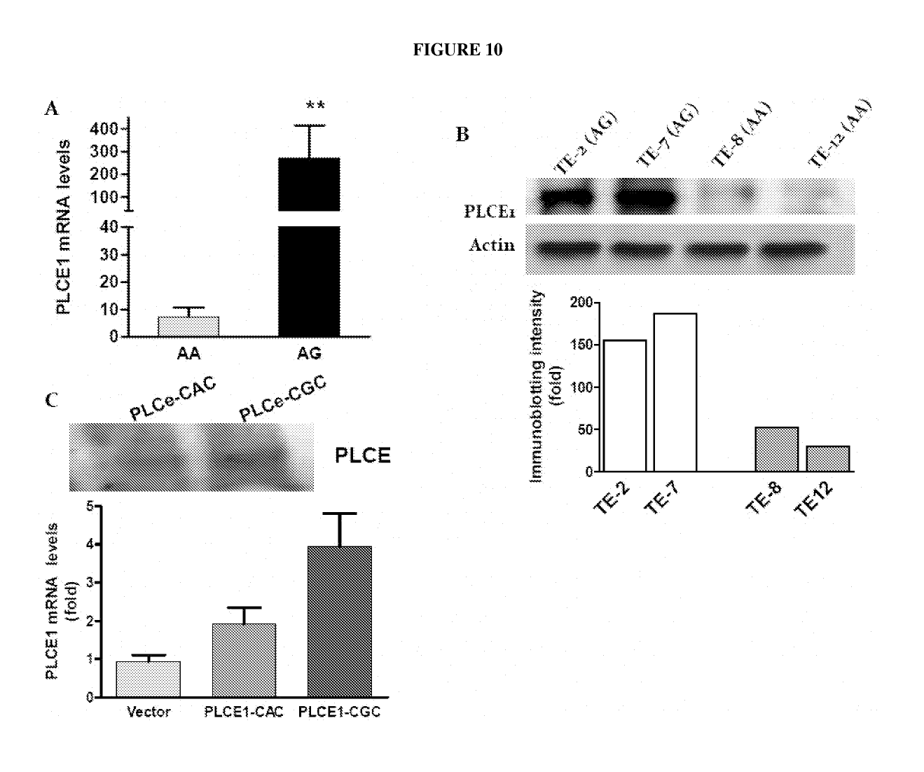

To investigate whether the G allele of the PLCE gene SNP at position 5780 alters PLCE expression, we determined PLCE genotypes and mRNA levels in 13 human esophageal squamous cancer cell lines (TI-1, TE-2, TE-7, TE-8, TE-12, G5, HCE4, HCE7, EC171, EC8712, EC109, EC9706, SHEEC). Six SCC cell lines (G5, TE-8, TE-12, HCE4, EC171, EC8712) were AA, while the remaining 7 (TE-1, TE-2, TE-7, HCE7, EC109, EC9706, SHEEC) were AG for PLCE at position 5780. Quantitative RT-PCR (qRT-PCR) revealed that PLCE mRNA levels in the seven SCC AG cell lines increased approximately 37-fold relative to the 6 AA SCC cell lines (FIG. 10A) (271±144 in AG cells vs. 7.3±3.3 in AA cells, p<0.01). Increased PLCE mRNA levels also correlated with higher PLCE protein levels measured by immunoblotting (FIG. 10B). Interestingly, two immortalized human esophageal epithelial cell lines derived from normal cells, HET1A (AG allele) and HEEPic (AA allele) also exhibited a similar association pattern: cells with the AG allele expressed higher levels of PLCE mRNA (23.6 in HET1A vs. 3.6 in HEEPic) and protein (data not shown).

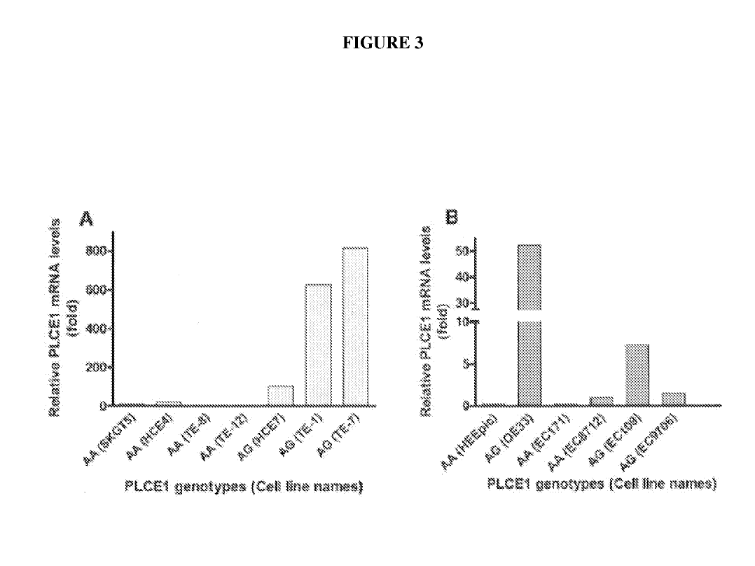

DNA and RNA were extracted from seven esophageal cancer cell lines, including SCC cells lines, including SCC cell lines TE-1, TE-2, TE-7, TE-8, TE-12, HCE-4, HCE-7 and an adenocarcinoma (EAC) cell line SDGT-5. Genotypes were assayed by PCR-based sequencing and mRNA levels were analyzed by quantitative real-time RT-PCR (qRT-PCR). As shown in FIG. 3, PLCE1 mRNA was significantly increased in the cancer cell lines that were heterozygous of PLCE1 (AG). Normal esophageal cells HEEPic (AA) has a very low level of PLCE1, but adenocarcinoma cell line OE33 and EC109 (derived from Chinese patient) was AG and PLCE 1 mRNA was very high. See FIG. 3B.

### Example 3

### PLCE1 Expression at mRNA and Protein Levels is Increased in Esophageal Cancers

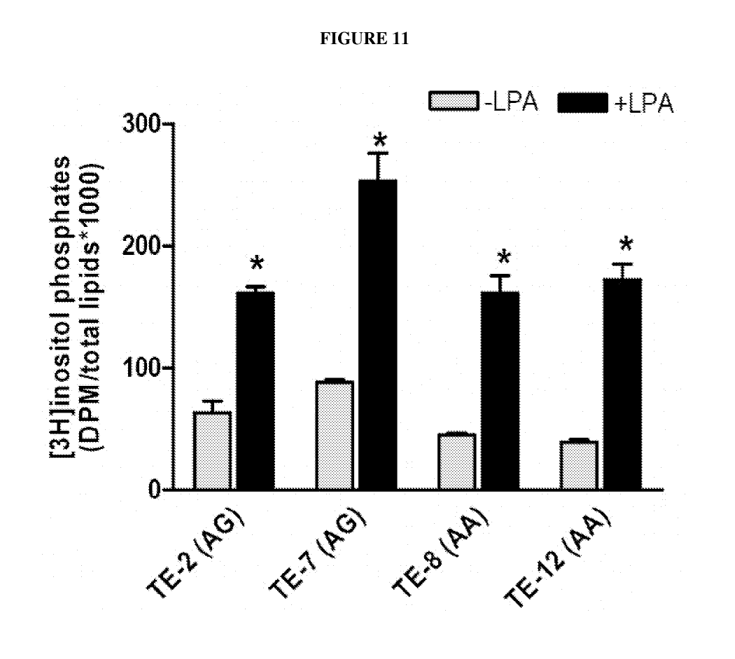

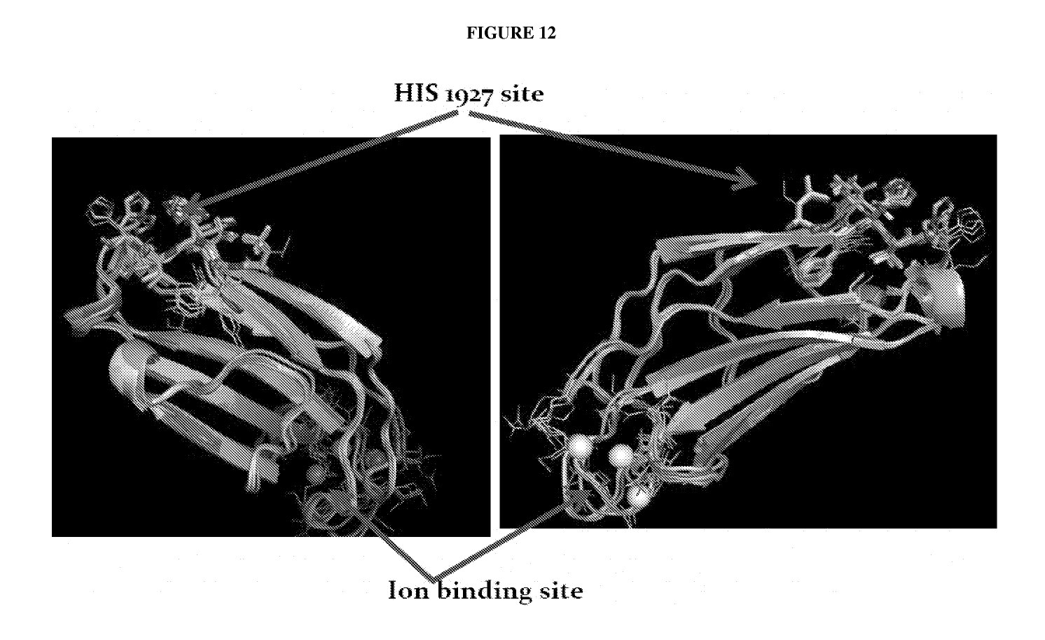

We then examined whether elevated PLCE mRNA and protein levels correlated with PLCE enzyme activity. For these experiments, we chose the two AG cell lines (TE-7 and TE-2) that expressed the highest levels of PLCE and two AA cell lines (TE-8 and TE-12) with the lowest levels of PLCE mRNA and protein. Consistent with findings for protein levels, endogenous PLCE baseline activity was nearly twice as high in the two AG cell lines than in the two AA cell lines (76±20 vs. 42±7, p<0.05). Previous studies have demonstrated that overexpression of activated small Rho family GTPases leads to marked elevation of intracellular inositol phosphate accumulation, and PLCE is a direct effector of activated Rho18. Similarly, the expression of G protein α-subunits Gα 12 and Gα 13 resulted in PLCE-dependent accumulation of inositol phosphates. Therefore, agonists of G Protein coupled receptors that couple to Gα 12 and Gα 13, such as LPA (1-Oleoyl-L-α-lysophosphatidic acid), activate PLCE in a Rho-dependent manner. To determine the response of PLCE activity to LPA in cells containing different alleles (AA vs AG), we treated the cells with LPA and surprisingly found that AG cells exhibited only 2.5-fold induction of PLCE activity, whereas AA cells were induced 4-fold (FIG. 11) (p<0.01). This finding may have been due to the failure of adapted AG allele cells to fully activate PLCE after interacting with harmful environmental factors (e.g., bile acids, bacterial infection, carcinogens, or other stressors), which reduces subsequential induction of cytokine and chemokine and development of inflammation in esophageal epithelium as a defensive response to the environmental detrimental stimulation. As a consequence, the lack of fully activated PLCE enzyme could cause epithelial cells to produce more PLCE mRNA and protein as a compensatory response through a feedback mechanism. From analysis of homology modeling of the PLCE C2 domain structure, we observed that changing His1927 to Arg in the C2 domain may affect protein-protein interaction and/or lipid recognition, but is unlikely to have an impact on ion binding by this enzyme (FIG. 12). However, further investigations using mutant allele plasmids are needed to confirm this modeling result.

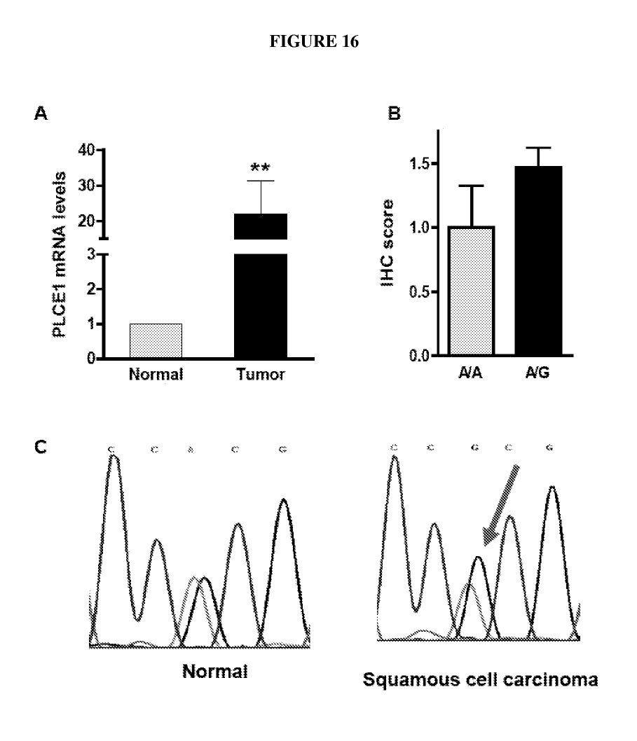

PLCE-mediated cell growth promotion has been reported in various cell types, and the mitogenic effect of PLCE may facilitate cancer progression. We measured PLCE mRNA levels in 26 primary human esophageal SCCs and adjacent normal esophageal epithelial tissues by quantitative RT-PCR. As shown in FIG. 16A, PLCE mRNA levels were significantly higher in esophageal SCCs than in adjacent normal tissues (normal, 1.0 vs. SCC, 22.0; p<0.01). Consistent with these mRNA expression levels, SCC tissues with heterozygous AG expressed 1.5-fold higher protein levels than did homozygous AA tissues (1.5±0.1 vs. 1.0±0.3, p<0.05) (FIG. 16B), assayed by immunohistochemical staining using an anti-PLCE antibody. Interestingly, allelic imbalance analysis showed an increased G allele copy number in 44% (15/34) of AG SCC tissues when compared to matching normal esophageal control tissues (FIG. 16C). These findings suggest that G allele is associated with overexpression of PLCE in primary SCC tissues.

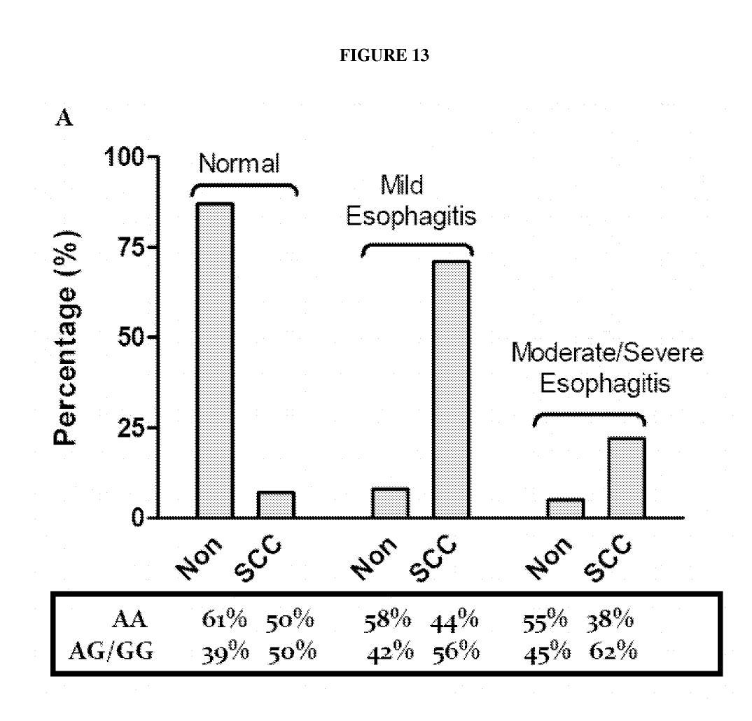

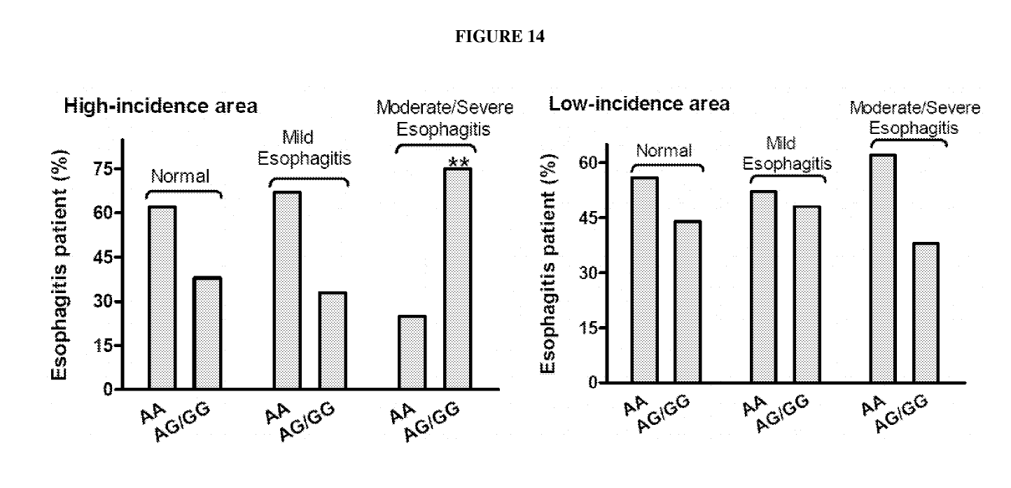

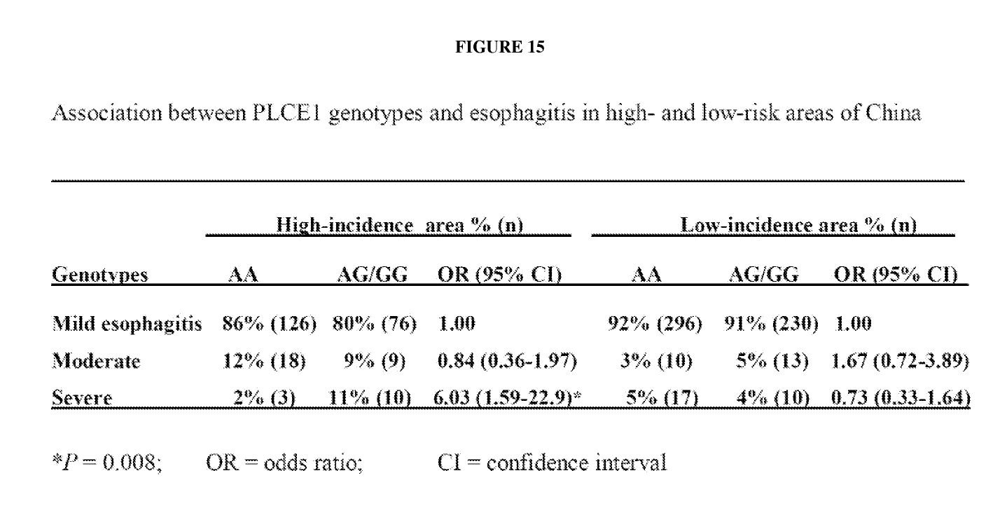

Overexpression of PLCE protein and enhancement of PLCE enzyme activity have been reported to activate PKC and induce elevation of intracellular calcium levels24, leading to cytokine- or chemokine-mediated inflammation in local tissues. Furthermore, an association between esophagitis and the development of esophageal squamous cell cancer has been recognized and documents previously. To investigate any possible association between A5780G and esophageal inflammation, we correlated SNP genotypes with presence or absence of esophagitis in individuals with cancer (SCC) and without cancer (Non-SCC). 52 (89.7%) of the 58 SCC patients exhibited various degrees of esophagitis (mild, moderate, or severe), whereas only 1517 (14.3%) of the 10,614 non-SCC subjects had any esophagitis, determined by endoscopic examination and confirmed by histophathology (P<0.0001). Importantly, the severity of esophagitis was associated with the AG/GG allele in these SCC patients. Eight (62%) of the 13 SCC patients with moderate or severe esophagitis had the AA genotype (FIG. 13). However, when non-SCC individuals with esophagitis were classified into high- or low-incidence areas for esophageal cancer, a significant association between severe esophagitis and the G allele of the PLCE gene were observed: 77% of the severe esophagitis individuals in high-incidence areas had AG/GG genotypes (FIG. 14), vs. only 37% of these subjects in low-incidence areas (FIG. 14) (OR 6.03 with 95% CI 1.59-22.9 vs. OR 0.74 with 95% CI 0.33-1.64; p=0.008; FIG. 15). These data support the hypothesis that the interaction of potential environmental factors with PLCE, particularly in the individuals with AG or GG allele, not only exists in high-incidence areas for esophageal cancer development in China, but also correlates with the severity of esophagitis.

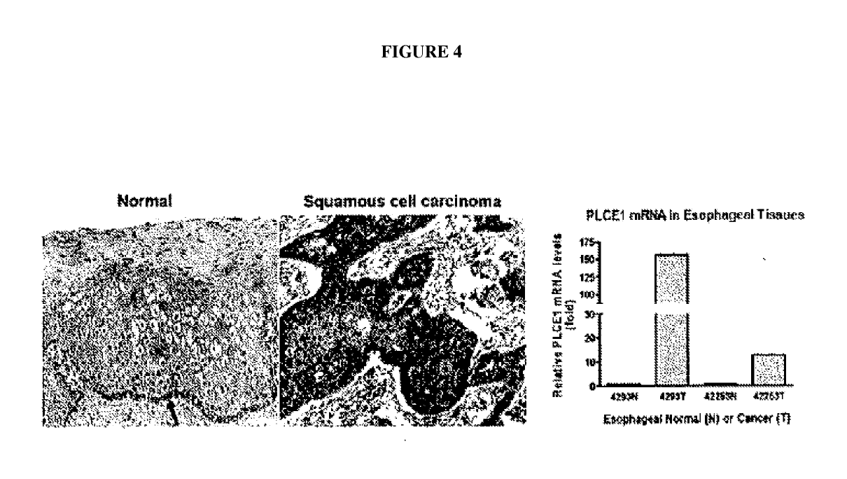

To determine the role of PLCE1 in esophageal carcinogenesis, we detected PLCE1 expression in esophageal squamous carcinomas at protein and mRNA levels by immunohistochemical staining and qRT-PCR, respectively, and compared to the normal epithelium. PLCE1 protein was overexpressed in cancer tissues although it was also slightly expressed in normal epithelial cells (basal cells). See FIG. 4. Moreover, qRT-PCR showed that PLCE1 mRNA was significantly increased in the 2 cancer tissues compared to their normal tissues. See FIG. 4.

We have found that about 40% of the G allele in the PLCE1 gene was gained (i.e. allelic imbalance) in esophageal squamous carcinomas than their adjacent normal tissues through screening about 30 pairs normal and SCC tissues. Additionally, using quantitative real-time RT-PCR, PLCE1 mRNA was significantly increased in the 2 SCC, compared to the normal control. See FIG. 4. These data illustrate that the G allele imbalance causes an increase of PCLE1 gene expression.

### Example 4

### Materials and Methods

Frozen tissues from 58 esophageal SCCs and white blood cell DNA from 10,614 non-cancer subjects were collected from an ongoing hospital-based SCC and EAC case-control study, involving multiple hospitals throughout high- and low-risk areas for esophageal SCC in China since 2007. The genotypes of PLCE in non-SCC patients were based on TaqMan genotyping methods. All subjects had undergone esphagogastroduodenoscopy, and non-cancer subjects with esophagitis were determined by endoscopic examination and histopathology. The diagnosis of the degree of esophagitis was made by at least three pathologists. All procedures were conducted according to Declaration of Helsinki principles and had been approved by respective institutional review boards.

Genomic DNA was extracted from the following materials: SCC and adjacent normal esophageal epithelia, esophageal SCC cell lines (TE-1, TE-2, TE-7, TE-8, TE-12, G5, HCE4, HCE7, EC171, EC8712, EC109, EC9706, SHEEC) and non-transformed esophageal epithelial cell lines (HET1A, HEEpic) using a DNA extraction kit (QIAGEN, Valencia, Calif.). PCR for PLCE exon 26, in which the C2 domain is located, was performed using the following primers: forward (Ex26F): 5′-TGTTCTTGGGATTCCTTTGC-3′ (SEQ ID NO:8) and reverse (Ex26R): 5′-TGCTTCTTAATTCAACTTCTTTATAGG-3′ (SEQ ID NO:9). The PCR product was directly sequenced using an ABI sequencing system (Applied Biosystems, Inc. Foster City, Calif.). Allelic imbalance was analyzed using Mutation Surveyor software (Softgenetics, College Station, Pa.).

Total RNA was extracted from human esophageal cancer cell lines and tissues, samples were then subjected to reverse transcription; cDNA was used for quantitative PCR analyses of PLCE expression at the mRNA level. The following primers that cover the C2 domain were used for quantitative PCR analysis: forward 5′-TGTGGAACGAGCAGTTTCTG-3′ (SEQ ID NO:10) and reverse 5′-ATCGAAGAGGCTGACATGGT-3′ (SEQ ID NO:11). Cell lysate was made from esophageal cancer cell lines, followed by immunoblotting and probing with anti-PLCE antibody. PLCE immunohistochemical staiing was performed, and immunohistochemical staining was scored by at least three pathologists using the following criteria: 0, no staining or staining area was less than 10%; 1, positive area was between 10% and 50%; and 2, positive are was more than 50%.

PLCE enzymatic activity was determined by measurement of total [³H]inositiol phosphates accumulation in esophageal cancer cells containing AG and AA alleles (TE-2, -7, -8 and -12). 1-Oleoyl-L-α-lysophosphatidic acid (LPA) was purchased from Sigma Aldrich and dissolved in water containing 1.0% fatty acid-free bovine serum albumin. Briefly, cells were seeded in a 24-well plate at a density of 200,000 cells per well. 18 hrs later, medium was replaced with inositol- and serum-free DMEM containing 1 μCi/well [³H]myo-inositol. Phosphlipase C activity was quantified 16 h after labeling by incubation in inositol-free DMEM containing 10 mM liCl, either in the absence of LPA or in the presence of 10 μM LPA. The reaction was stopped after 30 min by aspiration of the medium and addition of an ice-cold buffer containing 0.6 M perchloric acid and 0.2 mM IP₆. After neutralization with buffer containing 1 M K2CO3 and 40 mM EDTA, the accumulation of [3H] inositol phosphates was quantified by Dowex chromatography. 0.1% Triton X-100/0.1 M NaOH was added to cells in each well to determine total lipids. Each experiment was performed in triplicate, and these triplicate experiments were also repeated independently three times.

1. A method for determining whether a subject has an esophageal or esophageal-related disorder, or a predisposition for an esophageal or esophageal-related disorder, comprising
(a) providing a nucleic acid-containing sample obtained from a subject; and
(b) determining whether the sample comprises an esophageal marker, wherein the marker is SEQ ID NO:1 or a fragment thereof,
wherein the presence of the marker indicates that the subject has an esophageal or an esophageal-related disorder, or a predisposition for an esophageal or esophageal-related disorder.

2. The method of claim 1, comprising further determining the presence of at least one other biomarker selected from the group consisting of TFF2, HE4, LGALS3, IL1RN, TRIP133, FIGNI1, CRIP1, S100A4, EXOSC8, EXPI, BRRN1, NELF, EREG, TMEM40 and TMEM109.

3. The method of claim 1, wherein the esophageal disorder is selected from the group consisting of esophageal cancer and esophagitis.

4. (canceled)

5. (canceled)

6. The method of claim 3, wherein the esophageal disorder is esophagitis, and wherein the subject does not have cancer.

7. The method of claim 1, wherein the esophageal-related disorder is a cancer selected from the group consisting of head and neck cancer, throat cancer, gastric cancer, and mouth cancer.

8. The method of claim 1, wherein the marker is detected by:
(a) amplifying a nucleic acid comprising the marker; and
(b) detecting the amplified nucleic acids, thereby detecting the marker.

9. The method of claim 8, wherein the marker is detected by sequencing.

10. The method of claim 8, wherein the marker is amplified using a pair of primers comprising the sequences selected from the group consisting of SEQ ID NO:8 and SEQ ID NO:9, SEQ ID NO:10 and SEQ ID NO:11, and SEQ ID NO:12 and SEQ ID NO:13.

11. The method of claim 8, wherein the amplified nucleic acids are detected by hybridizing an oligonucleotide probe to the amplified product.

12. The method of claim 11, wherein the probe is labeled with a detectable label.

13. The method of claim 11, wherein the probe is an oligonucleotide comprising SEQ ID NO:1 or a fragment thereof.

14. The method of claim 1, wherein the fragment comprises between 10 and 100 contiguous nucleotides of SEQ ID NO:1, and wherein the contiguous sequence contains the guanine at position 401 of SEQ ID NO:1.

15. The method of claim 14, wherein the fragment is selected from the group consisting of SEQ ID NO:2 and SEQ ID NO:14.

16. A method for determining whether a subject has an esophageal or esophageal-related disorder, or a predisposition for an esophageal or esophageal-related disorder, comprising:
(a) contacting an antibody that specifically binds to a polypeptide encoded by the SEQ ID NO:1, or a fragment thereof, with a sample, thereby forming a complex between the antibody and the polypeptide; and
(b) detecting the presence of the complex, thereby detecting the marker,
wherein the presence of the marker indicates that the subject has an esophageal or esophageal-related disorder, or a predisposition for an esophageal or esophageal-related disorder.

17. The method of claim 16, wherein the fragment comprises between 15 and 86 amino acids of SEQ ID NO:3, wherein the contiguous sequence contains the arginine at position 53 of SEQ ID NO:3.

18. The method of claim 16, wherein the fragment is selected from the group consisting of SEQ ID NO:3, SEQ ID NO:4 and SEQ ID NO:7.

19. The method of claim 16, wherein the antibody is labeled with a detectable label.

20-38. (canceled)

39. A kit comprising:
(a) nucleic acid sample collecting means;
(b) means for determining the presence of a esophageal marker in a nucleic acid; and
(c) a control sample comprising polymorphic DNA, wherein the polymorphic DNA is rs2274223 or a fragment thereof.

40. A kit comprising
(a) sample collecting means;
(b) means for determining the presence of an esophageal marker in a protein; and
(c) a control sample comprising a polypeptide encoded by SEQ ID NO:1, or a fragment thereof.

41. An isolated peptide consisting of SEQ ID NO:4.


SEQUENCE LISTING (14 sequences)

SEQ ID NO: 1 - DNA, 801 bp, Artificial Sequence
  Sequence corresponding to dbSNP accession no. rs2274223.
atggtctcaatctcctgacctcgtgatctgctcacctcagcctcccaaagtgctgggattacaggcataagccaccgcgcccagccctacaatcacttactttttaaacagttttattcatcattcactttgtccattccagtgttcttgggattcctttgcagagggaagcagtgaggtgcagaggttgtctttcttttttatcctcggtgactttgatcccttttgtctccctcaccctagattgtctctggtcagaatgtgtgccccagtaatagcatgggaagcccgtgcattgaagtcgacgtcctgggcatgcctctggacagctgccatttccgcacaaagcccatccatcgaaacaccctgaaccccatgtggaacgagcagtttctgttccgcgttcacttcgaagatcttgtatttcttcgttttgcagttgtggaaaacaatagttcagcggtaactgctcagagaatcattccactgaaagctttaaaacgaggtagaataaaattgtccaaatgttaataattgttgtagctaggtgatggatgccagaatttccttatactcttctctcttttctgtttgacattttcctataaagaagttgaattaagaagcaaaaggaaatacaaattggagcacttttgaagcacatctcaaaagaagtttttcttttacattttttaagaatctgaggtattaacaatatcccttgtaaagtacagcctcacccatgcttgctgatgtcaggagcagtggttgttgctacccacggcactgctggcagtttga

SEQ ID NO: 2 - DNA, 33 bp, Artificial Sequence
  Fragment of SEQ ID NO1.
gagcagtttctgttccgcgttcacttcgaagat

SEQ ID NO: 3 - protein, 87 aa, Artificial Sequence
  Peptide encoded by fragment of SEQ ID NO1.
IVSGQNVCPSNSMGSPCIEVDVLGMPLDSCHFRTKPIHRNTLNPMWNEQFLFRVHFEDLVFLRFAVVENNSSAVTAQRIIPLKALKR

SEQ ID NO: 4 - protein, 11 aa, Artificial Sequence
  Peptide encoded by fragment of SEQ ID NO1.
EQFLFRVHFED

SEQ ID NO: 5 - DNA, 33 bp, Artificial Sequence
  Wild type sequence of a fragment of SEQ ID NO1.
gagcagtttctgttccacgttcacttcgaagat

SEQ ID NO: 6 - protein, 11 aa, Artificial Sequence
  Peptide encoded by SEQ ID NO5.
EQFLFHVHFED

SEQ ID NO: 7 - protein, 99 aa, Artificial Sequence
  Peptide encoded by fragment of SEQ ID NO1.
VYSLTIVSGQNVCPSNSMGSPCIEVDVLGMPLDSCHFRTKPIHRNTLNPMWNEQFLFRVHFEDLVFLRFAVVENNSSAVTAQRIIPLKALKRGYRHLQL

SEQ ID NO: 8 - DNA, 20 bp, Artificial Sequence
  Primer oligonucleotide.
tgttcttgggattcctttgc

SEQ ID NO: 9 - DNA, 27 bp, Artificial Sequence
  Primer oligonucleotide.
tgcttcttaattcaacttctttatagg

SEQ ID NO: 10 - DNA, 20 bp, Artificial Sequence
  Primer oligonucleotide.
tgtggaacgagcagtttctg

SEQ ID NO: 11 - DNA, 20 bp, Artificial Sequence
  Primer oligonucleotide.
atcgaagaggctgacatggt

SEQ ID NO: 12 - DNA, 20 bp, Artificial Sequence
  Primer oligonucleotide.
tgttcttgggattcctttgc

SEQ ID NO: 13 - DNA, 27 bp, Artificial Sequence
  Primer oligonucleotide.
tgcttcttaattcaacttctttatagg

SEQ ID NO: 14 - DNA, 261 bp, Artificial Sequence
  Fragment of SEQ ID NO1.
attgtctctggtcagaatgtgtgccccagtaatagcatgggaagcccgtgcattgaagtcgacgtcctgggcatgcctctggacagctgccatttccgcacaaagcccatccatcgaaacaccctgaaccccatgtggaacgagcagtttctgttccgcgttcacttcgaagatcttgtatttcttcgttttgcagttgtggaaaacaatagttcagcggtaactgctcagagaatcattccactgaaagctttaaaacga